(19) United States
(12) Patent Application Publication
(10) Pub. No.: US 2024/0382722 A1
(43) Pub. Date: Nov. 21, 2024

(54) Drain Electrode

(71) Applicant: UNM Rainforest Innovations, Albuquerque, NM (US)

(72) Inventor: Andrew Phillip Carlson, Albuquerque, NM (US)

(73) Assignee: UNM Rainforest Innovations, Albuquerque, NM (US)

(21) Appl. No.: 18/693,931

(22) PCT Filed: Sep. 20, 2022
(86) PCT No.: PCT/US22/44153

§ 371 Date: Mar. 20, 2024

Related U.S. Application Data
(60) Provisional application No. 63/246,284, filed on Sep. 20, 2021.

(51) Int. Cl. A61M 27/00; A61M 25/00

(52) U.S. Cl. CPC A61M 27/006; A61M 25/007; A61M 2205/04; A61M 2205/3317; A61M 2205/3344; A61M 2210/0693; A61M 2230/10

(57) ABSTRACT

A method for monitoring patients undergoing cSDH evacuation for spreading depolarization comprising the steps of: placing a subdural electrode strip and drain tube having a distal tip on the cortex of the patient, and a method and device wherein, at the time of surgery, a subdural assembly comprising a drainage tube and an electrode strip is placed between the brain and skull of a patient undergoing cSDH evacuation.

### RELATED APPLICATIONS

This is a 371 National Phase of PCT/US2022/044153 filed on 20 Sep. 2022, which claims priority to U.S. Provisional Application No. 63/246,284, filed on 20 Sep. 2021, both of which are incorporated herein in their entirety.

### BACKGROUND OF THE INVENTION

Most patients with chronic subdural hematoma (cSDH) recover after surgical evacuation with a straight-forward course. There is a subset of patients who develop transient and fluctuating deficits not explained by seizures, stroke, or mass effect after evacuation.

### BRIEF SUMMARY OF THE INVENTION

In one embodiment, the present invention provides a method and device wherein, at the time of surgery, a 1×6 subdural electrode strip is placed on the cortex parallel to the subdural drain in patients undergoing cSDH evacuation.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1A shows the cortical electrode placement for an embodiment of the present invention.

FIG. 1B show an ElectroCochleoGraphy (ECoG) tracing of a typical cortical spreading depolarization (SD) where the upper six tracings show full-band ECoG to reveal the characteristic slow DC potential changes propagating between electrodes at approximately 2 mm/min (arrows) and the lower six tracings show data from the same electrodes but filtered at 0.5-50 Hertz, to emphasize the suppression of high frequency activity during propagation of the same event open (dashed arrows).

FIG. 2 shows a cortical electrode for an embodiment of the present invention.

FIG. 3 shows a bedside subdural drain, electrode and ICP sensor place by a bolt for environment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

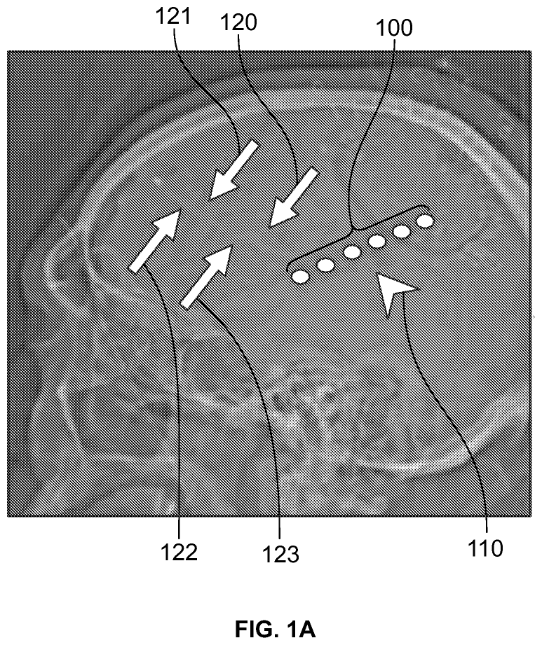

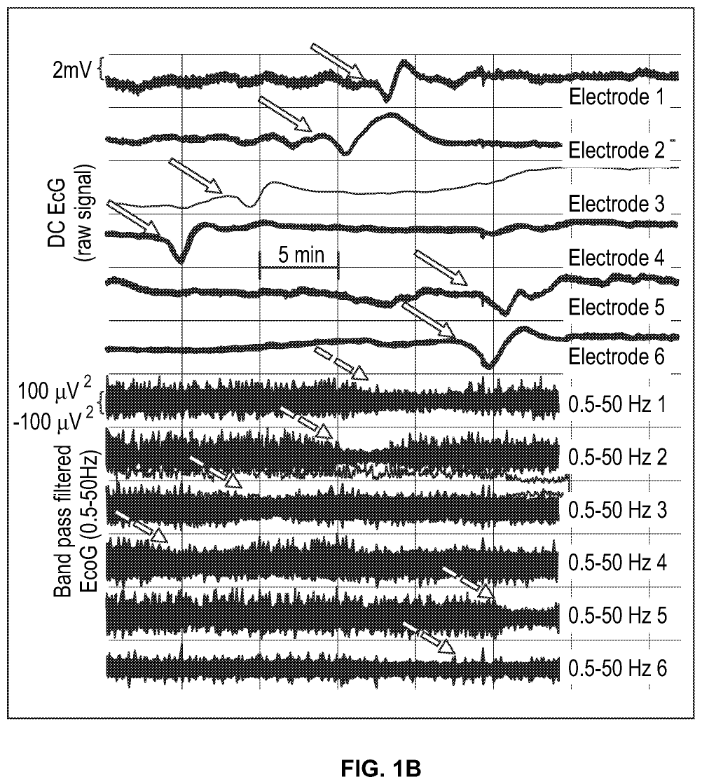

In one embodiment, as shown in FIG. 1, the present invention provides a method and device wherein, at the time of surgery, a 1×6 subdural electrode strip 100 is placed on the cortex parallel to the subdural drain in patients undergoing cSDH evacuation. For these patients, definitive SD was detected in 6 (15%) of 40 patients. Baseline and cSDH characteristics did not differ between patients with and without SD. More patients experienced postoperative neurological deterioration if they had SD (50%) compared to those without SD (8.8%; p=0.03). Only 2 patients in the entire cohort demonstrated early neurological deterioration, both of whom had SD. One of these cases demonstrated a time-locked new focal neurological deficit (aphasia) at the start of a series of multiple clusters of SD.

This is the first observation of SD occurring after cSDH evacuation. SD occurred at a rate of 15% and was associated with neurological deterioration. Thus, the present invention represents a novel method, device and mechanism for otherwise unexplained fluctuating neurological deficit after cSDH evacuation.

For a preferred embodiment concerning cortical electrode placement, subdural 1×6 platinum electrodes as indicated by arrowhead 110 may be placed onto the cortex parallel to the subdural drain as shown by arrows 120-125.

FIG. 1B shows an ECoG tracing of typical cortical SD. The upper 6 tracings show full-band ECoG to reveal the characteristic slow DC potential changes propagating between electrodes at approximately 2 mm/min (arrows). The lower 6 tracings show data from the same electrodes but filtered at 0.5-50 Hz, to emphasize the suppression of high-frequency activity during propagation of the same event (dashed arrows).

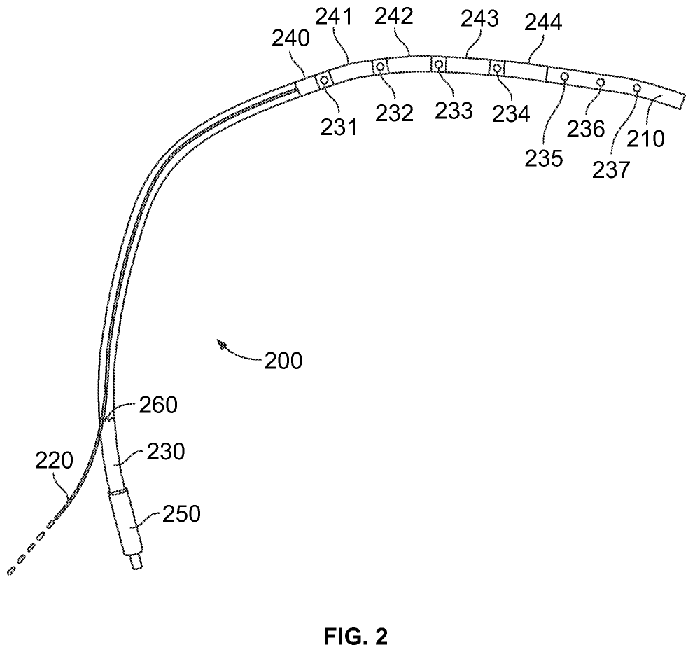

As shown in FIG. 2, in another embodiment, the present invention provides an electrode and drain combination 200 having a trimmable distal tip 210 that may be configured for accurate placement. Also provided is an electrode 220 that is inserted into the drainage tube 230 which has spaced-apart perforations 231-236. The two pieces, electrode 220 and drain tube 230, may be combined to form an assembly for treating a patient.

Proximately located from the tip is the electrode section which may include a plurality of electrical electrodes 240-244 comprising an electrode strip. In one preferred embodiment, six platinum electrodes may be used. Drainage tube 230 terminates with a luer lock adapter 250 as well as an access 260 to receive the electrode. In addition, perforated segments may be included on the drainage tube which are adapted to function as drainage holes.

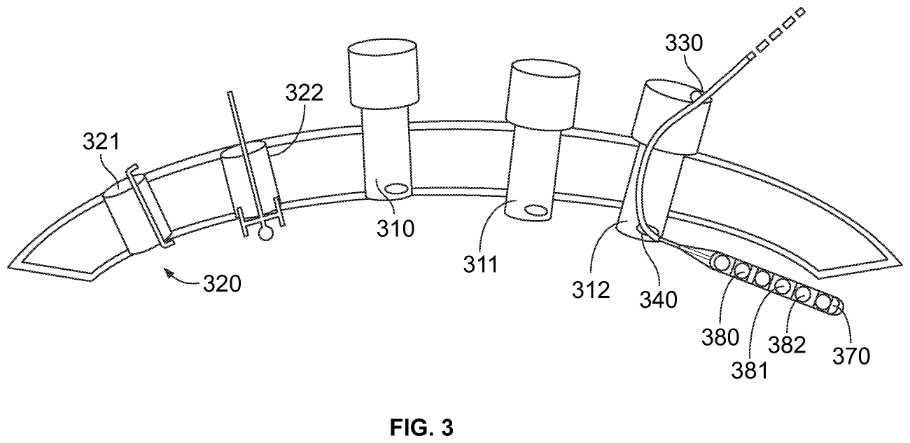

As shown in FIG. 3, one or more bolt channels 310-312 may be used to direct the electrode at an angle of 90°. In a preferred embodiment, the bolt channels are fully inserted with a 2 mm inset into the subdural space 320 to create a gap between the brain and skull (not shown). This may be accomplished by first making a drill hole 321 into the subdural space 320. The entire dura should be opened the entire hole width 322.

The bolt is designed to have a first or upper opening 330 adapted to receive the drainage tube and electrode from outside the patient and a second opening 340 adapted to direct the assembly into the gap created by the bolt. Lastly, an intracranial pressure (ICP) sensor 370 may be located at the tip along with a plurality of electrodes 380-382.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A method for monitoring patients undergoing cSDH evacuation for spreading depolarization comprising the steps of: placing a subdural electrode strip and drain tube having a distal tip on the cortex of the patient.

2. The method and device of claim 1 wherein the electrode strip includes a plurality of electrodes located inside said distal tip of said drain tube.

3. The method and device of claim 2 wherein said electrode strip includes six platinum electrodes.

4. The method and device of claim 2 wherein said drain tube includes a trimmable distal tip.

5. The method and device of claim 4 wherein said drain tube includes perforations that function as drainage holes along the distal portion of said tip.

6. The method and device of claim 5 further including one or more bolt channels configured to angularly direct said electrode strip and said drain tube.

7. The method and device of claim 5 further including one or more bolt channels configured to angularly direct said electrode strip and said drain tube at an angle of 90°.

8. The method and device of claim 7 wherein the bolt channels are fully inserted with a 2 mm inset into the subdural space.

9. The method and device of claim 8 further including an ICP sensor.

10. The method and device of claim 8 further including an ICP sensor located at said tip.

11. A method and device wherein, at the time of surgery, a subdural assembly comprising a drainage tube and an electrode strip is placed between the brain and skull of a patient undergoing cSDH evacuation.

12. The method and device of claim 11 wherein said electrode strip includes a plurality of electrodes located inside said distal tip of said drain tube.

13. The method and device of claim 12 wherein said electrode strip includes six platinum electrodes.

14. The method and device of claim 12 wherein said drain tube includes a trimmable distal tip.

15. The method and device of claim 14 wherein said drain tube includes perforations that function as drainage holes along the distal portion of said tip.

16. The method and device of claim 15 further including one or more bolt channels configured to angularly direct said electrode strip and said drain tube.

17. The method and device of claim 15 further including one or more bolt channels configured to angularly direct said electrode strip and said drain tube at an angle of 90°.

18. The method and device of claim 17 wherein the bolt channels are fully inserted with a 2 mm inset into the subdural space.

19. The method and device of claim 18 further including an ICP sensor.

20. The method and device of claim 18 further including an ICP sensor located at said tip.
